(19) United States
(12) Patent Application Publication
(10) Pub. No.: US 2012/0328760 A1
(43) Pub. Date: Dec. 27, 2012

(54) EMULSION COMPOSITION

(75) Inventors: Fumiko Harada, Kanagawa (JP); Yasushi Ichimi, Mie (JP); Osamu Mori, Kanagawa (JP)

(73) Assignee: AJINOMOTO CO., INC., Chuo-ku (JP)

(21) Appl. No.: 13/530,345

(22) Filed: Jun. 22, 2012

Related U.S. Application Data
(63) Continuation of application No. PCT/JP2010/073456, filed on Dec. 24, 2010.

(30) Foreign Application Priority Data
Dec. 24, 2009 (JP) 293395/2009

(51) Int. Cl. A23D 7/005; A23L 1/30; A23L 2/52

(52) U.S. Cl. 426/590; 426/602; 426/648

(57) ABSTRACT

An emulsion composition, which contains (A) an oil phase component containing an oil-soluble component and fats and oils, (B) polyglycerol fatty acid ester, and (C) an aqueous phase component, wherein the (A) fats and oils in the oil phase component have a fatty acid composition in a weight ratio of capric acid 20 to 97, lauric acid 28 to 6000, and myristic acid 11 to 2100, relative to caprylic acid 100, has a high emulsion stability even after long-term preservation, which is sensorially superior, shows a high transparency when added to an aqueous phase system, and suppresses decomposition of an oil-soluble component having physiological functions such as a capsinoid compound.

### CROSS REFERENCES TO RELATED APPLICATIONS

This application is a continuation of International Patent Application No. PCT/JP2010/073456, filed on Dec. 24, 2010, and claims priority to Japanese Patent Application No. 2009-293395, filed on Dec. 24, 2009, both of which are incorporated herein by reference in their entireties.

### BACKGROUND OF THE INVENTION

1. Field of the Invention

The present invention relates to emulsion compositions suitable for addition of an oil-soluble component such as a capsinoid compound and the like to an aqueous phase.

2. Discussion of the Background

A less pungent chili pepper, “CH-19 Sweet”, which is a nonpungent fixed variety of chili pepper selected and fixed by Yazawa et al. has been reported to contain a large amount of pungent-free novel capsinoid compounds. Such compounds belonging to capsinoid compounds (fatty acid ester of vanillyl alcohol, capsiate, dihydrocapsiate, etc., hereinafter sometimes to be simply referred to as “capsinoid” or “capsinoid compound”) are different from capsaicinoid (capsaicin, dihydrocapsaicin, etc.), which is a pungent component of chili pepper, and do not have a pungent taste. However, they have been reported to show an immunity enhancing action, an energy metabolism activation action, and the like (see JP-A-H11-246478, which is incorporated herein by reference in its entirety), and are expected to be applicable in the future.

Capsinoid compounds are highly unstable since an ester bond present between the vanillyl group and the fatty acid side chain is easily hydrolyzed. To enable addition to aqueous foods and drinks such as beverage and the like, a technique is required for blending or dispersing the compound in the aforementioned aqueous medium while maintaining the stability thereof. As such techniques, one including emulsifying capsinoid containing oil with various emulsifiers has been reported (see JP-A-2003-192576, which is incorporated herein by reference in its entirety).

In addition, a technique for improving the stability of a capsinoid compound in an emulsion composition, including adding, during preparation of a capsinoid-containing emulsion composition, a thickener to an oil phase containing the capsinoid compound to enhance the viscosity of fats and oils in the oil phase has been reported (see JP-A-2007-269714, which is incorporated herein by reference in its entirety).

For emulsion compositions, turbidity is also important, since an emulsion composition with high turbidity makes it difficult to provide an aqueous solution with high transparency by adding the same to an aqueous phase system. While the turbidity of an emulsion composition is evaluated by transmission rate and average particle size of the emulsion composition, in general, an emulsion composition having a smaller average particle size shows a lower turbidity, and when the average particle size is 100 nm or below, a transparent emulsion composition tends to be provided. A technique for transparently emulsifying an oil phase containing a flavor, dye, oil-soluble vitamin, edible fats and oils, wax and the like by using particular polyglycerol fatty acid ester and polyvalent alcohol has also been reported (see JP-A-62-250941, which is incorporated herein by reference in its entirety).

While the above-mentioned JP-A-2003-192576 shows stability of a capsinoid-containing emulsion composition in an acidic region, when an aqueous drink added with the emulsion composition is in fact prepared, the capsinoid in the emulsion composition is gradually decomposed during ambient temperature preservation, thus posing a problem.

In addition, the emulsion composition described in the above-mentioned JP-A-2007-269714 requires an increased viscosity of the oil phase by addition of a thickener. Moreover, the emulsion composition prepared as mentioned above shows preservation stability of capsinoid, but no consideration has been made as to the transparency thereof.

Furthermore, the above-mentioned JP-A-62-250941 does not provide quantitative consideration of the transparency of the emulsion composition, and further emulsion stability has also been desired.

Thus, there remains a need for emulsion compositions is suitable for addition of an oil-soluble component such as a capsinoid compound and the like to an aqueous phase.

### SUMMARY OF THE INVENTION

The present invention, therefore, aims to provide an emulsion composition which is superior in the preservation stability of an oil-soluble component unstable in the aqueous phase, including a capsinoid, and which is superior in transparency when added to an aqueous phase system.

Accordingly, it is one object of the present invention to provide novel emulsion compositions suitable for addition of an oil-soluble component such as a capsinoid compound and the like to an aqueous phase.

It is another object of the present invention to provide novel emulsion compositions which contain a capsinoid compound.

It is another object of the present invention to provide novel emulsion compositions which contain a capsinoid compound, which provide improved stability of the capsinoid compound.

It is another object of the present invention to provide novel emulsion compositions which contain a capsinoid compound, which exhibit improved transparency.

These and other objects, which will become apparent during the following detailed description, have been achieved by the inventors' discovery that the stability of the oil-soluble component becomes high when the fatty acid composition of the oil phase is set to a particular ratio. In addition, it has been clarified by quality evaluation of the transparency, emulsion stability, preservation stability of the oil-soluble component such as capsinoid and the like, and the like that the presence of caprylic acid, capric acid, lauric acid, and myristic acid in a particular ratio range in the fatty acid composition of fats and oils in the oil phase component is important.

Accordingly, the present invention provides at least the following.

(1) An emulsion composition, comprising

(A) an oil phase component containing an oil-soluble component and fats and oils,

(B) polyglycerol fatty acid ester, and

(C) an aqueous phase component, wherein

(A) fats and oils in the oil phase component have a fatty acid composition in a weight ratio of capric acid 20-97, lauric acid 28-6000, and myristic acid 11-2100, relative to caprylic acid 100.

(2) The emulsion composition of the above-mentioned (1), comprising 1-2000 parts by weight of (A) oil phase component relative to 100 parts by weight of (B) polyglycerol fatty acid ester.

(3) The emulsion composition of the above-mentioned (1), wherein a water dispersion obtained by dispersing the composition in water such that the (A) oil phase component is contained in 0.25 wt % shows a light transmission rate of not less than 90% at a wavelength of 600 nm.

(4) The emulsion composition of the above-mentioned (1), wherein (B) polyglycerol fatty acid ester comprises, as a main component, polyglycerol monomyristate which is an ester of polyglycerol containing not less than 30 wt % of polyglycerol having a degree of polymerization of not less than 10 and fatty acid containing not less than 90 wt % of myristic acid.

(5) The emulsion composition of any of the above-mentioned (1)-(4), wherein (A) oil-soluble component comprises one or more kinds selected from capsinoid compounds.

(6) The emulsion composition of the above-mentioned (5), wherein one or more kinds selected from capsinoid compounds are selected from capsiate, dihydrocapsiate and nordihydrocapsiate.

(7) A food or drink comprising 0.001 wt %-10 wt % of the emulsion composition of any of the above-mentioned (1)-(6).

According to the present invention, an emulsion composition showing high emulsion stability after a long-term preservation, which is sensorially superior, affords a high transparency when added to an aqueous phase system, and suppresses decomposition of an oil-soluble component such as a capsinoid compound and the like contained in the composition can be provided.

### DETAILED DESCRIPTION OF THE PREFERRED EMBODIMENTS

While the present invention is explained in detail in the following, the scope of the present invention is not limited thereby.

The emulsion composition of the present invention comprises (A) an oil phase component containing an oil-soluble component and fats and oils, (B) polyglycerol fatty acid ester, and (C) an aqueous phase Component, wherein the fats and oils in the (A) oil phase component has a particular range of fatty acid composition.

### (A) Oil Phase Component.

In the present invention, (A) the oil phase component characteristically contains an oil-soluble component and fats and oils. As the oil-soluble component used in the present invention, an oil-soluble component useful for the body, or an oil-soluble component useful for utilization for foods and drinks or cosmetics are preferably used. Examples of the oil-soluble component include oil-soluble medicaments, oil-soluble vitamins such as liver oil, vitamin A, vitamin A oil, vitamin D₃, vitamin B₂ butyrate, fatty acid esters of ascorbic acid, natural vitamin E mixture, vitamin K, and the like; oil-soluble dyes such as paprika pigment, annatto pigment, tomato pigment, marigold pigment, β-carotene, astaxanthin, canthaxanthin, lycopene, chlorophyll, and the like; flavors such as orange oil, peppermint oil, spearmint oil, cinnamon oil, and the like; plant essential oils such as limonene, linalool, nerol, citronellol, geraniol, citral, l-menthol, eugenol, cinnamic aldehyde, anethole, perillaldehyde, vanillin, γ-undecalactone, and the like; physiologically active components such as coenzyme Q₁₀, α-lipoic acid, ω-3 fatty acid (Ω-linolenic acid, eicosapentaenoic acid, docosahexaenoic acid and the like), ω-6 fatty acid (linoleic acid, γ-linolenic acid and the like), phytosterol, and the like; and the like. An amount effective for pharmaceutical products, cosmetic agents, foods and drinks, and the like can be added. In the present invention, components easily reactive and unstable in general preparations and products due to hydrolysis, oxidative decomposition and the like, such as capsinoid compounds, oil-soluble vitamins (vitamin A, fatty acid esters of ascorbic acid and the like), ω-3 fatty acid and the like can be preferably used.

In the present invention, a capsinoid compound can be preferably used as the above-mentioned oil-soluble component. A capsinoid compound preferably refers to a fatty acid ester of vanillyl alcohol, and representative compounds include, but is not limited to, capsiate, dihydrocapsiate, nordihydrocapsiate confirmed as components contained in chili peppers, and further, also include fatty acid esters of various straight chain or branched chain fatty acids and vanillyl alcohol, which have a fatty acid chain length almost the same as capsiate and nordihydrocapsiate such as vanillyl decanoate, vanillyl nonanoate, vanillyl octanoate, and the like. Capsiate (hereinafter sometimes to be abbreviated as “CST”), dihydrocapsiate (hereinafter sometimes to be abbreviated as “DCT”) and nordihydrocapsiate (hereinafter is sometimes to be abbreviated as “NDCT”) can be represented by the following chemical formulas, respectively.

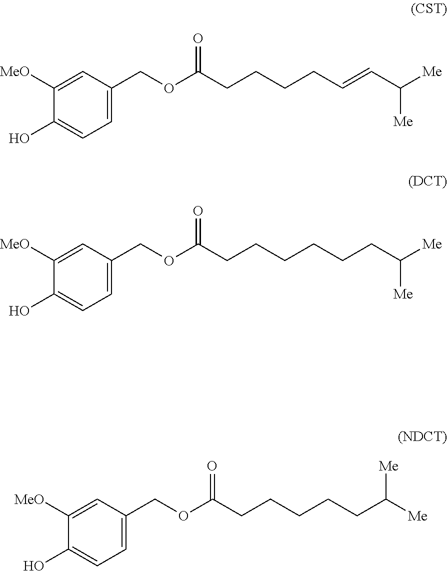

Since capsinoid compounds are contained in large amounts in a plant body belonging to the genus Capsicum (hereinafter to be referred to as “chili pepper”), it can be prepared by separation and purification from a plant body and/or fruit of a chili pepper. The chili pepper to be used for the purification is not particularly limited as long as it contains capsinoids, and a chili pepper derived from a native variety having a pungent taste represented by “NIKKO”, “GOSHIKI” and the like may be used. However, a chili pepper of a pungent-free variety is preferable. Particularly, pungent-free varieties such as “CH-19 Sweet”, “MANGANJI”, “FUSHIMI AMANAGA” and the like, green pepper, pepper, and the like can be preferably used, since they contain a capsinoid compound in large amounts. Particularly, “CH-19 Sweet”, which is a pungent-free variety, is more preferable, since it has a high content of the component. In the present specification, the term “CH-19 Sweet” means a group of varieties including “CH-19 Sweet” variety, and progeny variety derived from “CH-19 Sweet” and the like. Separation and purification of the capsinoid compound can be performed by solvent extraction, various chromatography methods such as silica gel chromatography and the like, high performance liquid chromatography for preparation and the like, which are well known to those of ordinary skill in the art, used singly or in appropriate combination. For example, the method described in JP-A-H11-246478 recited above can be used.

In addition, the above-mentioned capsinoid compound can also be synthesized, for example, by a transesterification reaction using the corresponding fatty acid ester and vanillyl alcohol as starting materials as described in JP-A-H11-246478 recited above. It can also be synthesized based on the structural formula thereof by other reaction methods well known to those of ordinary skill in the art. Furthermore, it can also be prepared easily by a synthesis method using an enzyme. For example, by the method described in JPA-2000-312598, and Kobata et al. (Biosci. Biotechnol. Biochem., 66 (2), 319-327, 2002), both of which are incorporated herein by reference in their entireties, a desired capsinoid compound can be easily obtained by utilizing a reverse reaction of lipase by using a compound such as fatty acid ester corresponding to the desired compound and/or triglyceride having the fatty acid and the like, and vanillyl alcohol as substrates.

When used for the preparation of the emulsifying composition of the present invention, the capsinoid compound may be any of the above-mentioned extracts and synthesized products, and a single capsinoid compound may be used or a mixture of two or more kinds thereof may be used. Moreover, the capsinoid compound to be used may contain a decomposition product thereof (free fatty acid, vanillyl alcohol and the like).

Examples of the fats and oils used in the present invention include plant-derived fats and oils such as soybean oil, coconut oil, rice oil, corn oil, palm oil, palm kernel oil, safflower oil, rape seed oil, olive oil, and the like; medium-chain saturated fatty acid triglyceride constituted of fatty acid containing saturated fatty acid having a carbon number of 6 to 10 (e.g., capric acid, caprylic acid, etc.) as a main constituent component and glycerol (hereinafter to be also referred to as “MCT”); animal-derived fats and oils such as beef tallow, lard, chicken fat, fish oil, and the like; fatty acids such as lauric acid, myristic acid, palmitic acid, oleic acid, and the like, mixtures thereof, and the like. These fats and oils can further contain, besides the above-mentioned oil-soluble components, one or more kinds of an antioxidant such as rosemary extract, butylhydroxyanisole (BHA), dibutylhydroxytoluene (BHT), t-butylhydroquinone (TBHQ), propyl gallate, and the like, specific gravity adjusting agents, and the like in combination.

In the present invention, it is important to select and use fats and oils such that the fats and oils in the oil phase component have a fatty acid composition in weight ratio of capric acid 20 to 97, lauric acid 28 to 6000, and myristic acid 11 to 2100, relative to caprylic acid 100. This range is preferable since the stability of the oil-soluble component such as a capsinoid compound and the like becomes higher as the ratio of capric acid, lauric acid, and myristic acid to caprylic acid becomes lower. However, when the ratio of capric acid, lauric acid, and myristic acid is lower than this range, emulsion stability of the emulsion composition is unpreferably degraded. In the present invention, it is preferable to use, in a weight ratio, capric acid 20 to 95, lauric acid 28 to 600, and myristic acid 11 to 1000, more preferably capric acid 31 to 95, lauric acid 30 to 250, and myristic acid 11 to 80, relative to caprylic acid 100.

The fatty acid composition of fats and oils can be measured by a known method and, for example, a conventional method such as gas chromatography and the like is used. The percentage of each peak area to the total of the chromatogram peaks obtained by this method is taken as the fatty acid composition. In the case of a mixture of fats and oils A and fats and oils B, the fatty acid composition is determined by the following formula.

{fatty acid composition of fats and oils A×mixing ratio of fats and oils A to the total as 1 (weight standard)}+{fatty acid composition of fats and oils B×mixing ratio of fats and oils B to the total as 1 (weight standard)}

### Emulsifier.

Preparation of the emulsion composition of the present invention is not particularly limited except use of an emulsifier containing (B) polyglycerol fatty acid ester, and various emulsifiers conventionally used for food and drink and the like can be used in combination with polyglycerol fatty acid ester. Examples of the emulsifier that can be used in combination include monoglycerol fatty acid esters, diglycerol fatty acid esters, triglycerol fatty acid esters, monoglycerol fatty acid ester derivatives, monoethyleneglycol fatty acid esters, diethylene glycol fatty acid esters, propylene glycol fatty acid esters, sorbitan fatty acid esters, polyoxyethylene sorbitan fatty acid esters, sucrose fatty acid esters, lecithin, quillai extract, and the like.

Considering the transparency of the obtained emulsion composition, (B) polyglycerol fatty acid ester used in the present invention is preferably a fatty acid ester of polyglycerol having an average degree of polymerization of not less than 6, considering the taste and flavor, it is preferably myristate. As the quality of the final product containing an emulsion composition, appearance, i.e., transparency or turbidity, is important, and particularly, taste, flavor and odor are also important for food and drink. As for flavor and odor, an off-flavor and an oxidized odor may be generated due to an aldehyde, ketone, and the like produced by reactions such as hydrolysis of fatty acid ester used as an emulsifier for an emulsion composition, oxidation of fatty acid and the like. The development of such off-flavor and oxidized odor varies depending on the fatty acid to be the starting material, and myristic acid is a fatty acid which shows less development of the aforementioned off-flavor and the like and is sensorially superior comparatively. Moreover, to achieve high transparency and good emulsion stability, one containing, as a main component, polyglycerol monomyristate, which is an ester of polyglycerol containing not less than 30 wt % of polyglycerol having a degree of polymerization of 10 or above and fatty acid containing not less than 90 wt % of myristic acid, is preferable. The degree of polymerization distribution and fatty acid composition of polyglycerol constituting polyglycerol fatty acid ester can be analyzed by high-performance liquid chromatography mass spectrometry (LC/MS).

### (C) Aqueous Phase Component.

The aqueous phase component for the emulsion composition of the present invention is used for the preparation of an oil-in-water type emulsion composition by emulsifying the above-mentioned oil phase. Where necessary, saccharides such as sugar, millet jelly, and the like; polyvalent alcohols such as glycerol, sorbitol, maltitol, erythritol, isomalt, propylene glycol, and the like; lower alcohols such as ethanol and the like; metal salts such as sodium chloride, potassium chloride, calcium chloride, and the like; water-soluble vitamins such as vitamin B₁, vitamin B₂, vitamin B₆, and the to like and salts thereof; water-soluble polymer compounds such as gum arabic, gum ghatti, gum tragacanth, guar gum, caraya gum, xanthan gum, pectin, alginic acid and salts thereof, carageenan, gelatin, casein, acrylic acid-alkyl methacrylate copolymer, cellulose derivatives (hydroxyethylcellulose, carboxymethylcellulose, and the like), modified starch, octenyl succinate starch, and the like; antioxidants such as catechin, vitamin C, sodium bisulfite, sodium erythorbate, tea extract, and the like; water-soluble dyes such as crocin (gardenia dye), anthocyanin, phycocyanin, and the like; and the like can also be contained as appropriate.

The pH of the aqueous phase component is preferably adjusted to the pH in the acidic range, more preferably pH 2 to 6. While the acidic substance used for adjusting to such a pH is not particularly limited, for example, organic acids such as citric acid, adipic acid, succinic acid, tartaric acid, lactic acid, fumaric acid, DL-malic acid, benzoic acid, gluconic acid, glucono delta-lactone, and the like and salts thereof; salts such as potassium carbonate, sodium hydrogen carbonate, sodium carbonate, sodium dihydrogen pyrophosphate, and the like, inorganic acids such as phosphoric acid and the like and salts thereof; and the like can be mentioned. Using these acidic substances, the pH of the emulsion composition is adjusted to the acidic range, whereby a capsinoid compound can be stably maintained for a long term.

In general, when the content ratio of the oil phase component to the emulsifier is low, an emulsion composition with high transparency can be obtained easily. In this case, the content of the oil phase component in the emulsion composition decreases, and the content of the oil-soluble component having various functions in the oil phase component also becomes small. In the emulsion composition of the present invention, the oil phase component is preferably contained at a ratio of 1 to 2000 parts by weight relative to 100 parts by weight of polyglycerol fatty acid ester. When the content ratio of the oil phase component and the emulsifier is high, the emulsion composition has a high viscosity to make handling thereof difficult. Thus, the aqueous phase component is preferably contained in not less than 20 wt %, more preferably not less than 40 wt %, based on the total weight of the emulsion composition. When the content ratio of the oil phase component and the emulsifier is too low, the emulsion becomes unstable. Therefore, the aqueous phase component is preferably contained in not more than 95 wt %, based on the total weight of the emulsion composition. In addition, the content of the oil-soluble component relative to the total amount of the emulsion composition is suitably 0.0001 wt % to 50 wt %. In this range, when the content of the oil-soluble component is high, the amount of the oil-soluble component contained in an aqueous food or drink, to which the emulsion composition is added, becomes preferably high. On the other hand, from the aspect of emulsion stability, the content of the oil-soluble component is preferable low, the content of the oil-soluble component relative to the total amount of the emulsion composition is further preferably 0.01 wt % to 10 wt %, more preferably 0.1 wt % to 2 wt %.

### Preparation of the Emulsion Composition.

For preparation of the emulsion composition of the present invention, an oil-soluble component such as a capsinoid compound and the like is dissolved in fats and oils preferably having the above-mentioned fatty acid composition, and the solution is subjected to a generally-used emulsification method of fats and oils such as mechanical emulsification, phase-transfer emulsification, liquid crystal emulsification, D-phase emulsification, and the like, whereby an emulsion composition can be prepared as appropriate. For example, in one preferable embodiment, firstly, an oil phase containing an oil-soluble component such as a capsinoid compound and the like, and an aqueous phase wherein the above-mentioned emulsifier is dissolved in water by heating are mixed, the emulsion composition is adjusted as necessary to a pH of 2 to 6 with the above-mentioned acidic substance, and mixed and homogenized using a homomixer, a colloid mill, a high-pressure homogenizer, a microfluidizer and the like to give an emulsion composition superior in the stability of the oil-soluble component such as a capsinoid compound and the like. When polyvalent alcohol is contained as an aqueous phase component, a D-phase emulsification method wherein an emulsifier is dissolved in polyvalent alcohol to form a D-phase, to which an oil phase component is gradually added for preliminary emulsification, and an aqueous phase component is added and mixed to allow emulsification can also be used preferably. To obtain a transparent emulsion composition, ultrafine emulsion particles having an average particle size of 100 nm or below need to be prepared, and emulsification using a super high-pressure homogenizer (microfluidizer) having a high shear force, a high-pressure homogenizer, and the like is preferable since an ultrafine emulsion composition can be obtained in a short time. In the present invention, the aforementioned average particle size is measured by a dynamic light scattering method.

The emulsion composition of the present invention is preferably transparent and free of turbidity when added to an aqueous phase system, in consideration of application to foods and drinks such as aqueous drinks and the like, liquid pharmaceutical products such as liquids and the like, and the like. The turbidity can be evaluated by a generally-used method and, for example, a method including measurement of light transmission rate at a wavelength of 600 nm can be mentioned. When the aforementioned transmission rate exceeds 90%, an aqueous product containing the emulsion composition shows transparency even after being placed in a transparent container, which is also sensorially preferable. When the emulsion composition of the present invention is added to and dispersed in water such that the oil phase containing an oil-soluble component is contained in 0.25 wt %, the obtained water dispersion preferably shows a light transmission rate of not less than 90% at a wavelength of 600 nm.

### Foods and Drinks and Aqueous Drinks Containing an Emulsion Composition of the Present Invention.

The emulsion composition of the present invention can be provided as a food or drink having a physiological action of an oil-soluble component such as a capsinoid compound and the like stably for a long term, by adding an adequate amount to foods and drinks such as aqueous drinks; milk products such as yoghurt and the like; frozen desserts such as ice cream and the like; confectioneries such as chocolate, candy, chewing gum, and the like; baked goods; seafood processed foods; meat processed foods; retort foods; frozen foods; and the like. Particularly, it is preferably provided as an aqueous drink containing a capsinoid and the like. An aqueous drink can be provided as a soft drink or carbonated drink by adding, where necessary, fruit juice, vitamins, amino acids, flavor, saccharides, acid, base, salts, and the like. In the present invention, moreover, the aforementioned food and drink may be provided as Food with health claims such as Food for specified health uses, Food with nutrient function claims and the like, or nutrition aid food. While the amount of the emulsion composition of the present invention relative to the aforementioned foods and drinks varies depending on the use object, kind, form, and the like of the foods and drinks, it is generally added within the range of 0.001 wt % to 10 wt %, based on the total weight of the food or drink. Furthermore, the total oil phase component derived from the emulsion composition is preferably added within the range of 0.00005 wt % to 5 wt %, more preferably 0.1 wt % to 0.3 wt %, relative to the aforementioned foods and drinks. When it is less than 0.00005 wt %, the stability of the oil-soluble component such as capsinoid etc. may not be preferable, and when it is not less than 5 wt %, the transparency, taste and flavor may not be preferable.

Moreover, in the present invention, an emulsion composition containing an oil-soluble medicament in the oil phase is dispersed in an aqueous carrier and the dispersion can be provided as an aqueous pharmaceutical product. As the aqueous carrier, water; lower alcohol such as ethanol and the like; polyvalent alcohol such as propanediol, 1,3-butanediol, glycerol, sorbitol, maltitol, and the like; and the like can be used. The aqueous pharmaceutical product of the present invention can be provided as liquid, elixir, emulsion, syrup, liniment, lemonade, lotion, and the like, and is suitable for oral administration, and parenteral administration such as injection, external, intragstrical administration, and the like. The amount of the emulsion composition to be added to an aqueous carrier, and the content of the oil-soluble component in the total amount of the aqueous pharmaceutical product are the same as those in the above-mentioned foods and drinks. In addition, additives generally used for aqueous pharmaceutical products such as a stabilizer, preservative, buffering agent, corrigent, flavoring agent, solubilizing agent, colorant, thickener, and the like can be contained as long as the characteristics of the present invention are not impaired.

Moreover, in the present invention, an emulsion composition containing an oil-soluble emollient, a skin cell activating component, and the like in the oil phase is dispersed in an aqueous carrier and the dispersion can be provided as a liquid cosmetic agent. As the aqueous carrier, those similar to the above-mentioned carriers can be used. The liquid cosmetic agent of the present invention can be provided as a skin cosmetic agent such as a skin lotion, skin milk, beauty essence, and the like; a makeup cosmetic agent such as a liquid-type foundation, eyeliner, mascara, and the like; a washing cosmetic agent such as a facial wash, cleansing agent, and the like, a body cosmetic agent such as a body lotion and the like; and the like. The amount of the emulsion composition to be added to an aqueous carrier, and the content of the oil-soluble component in the total amount of the cosmetic agent are the same as those in the above-mentioned foods and drinks. In addition, general starting materials and additives for cosmetic agents such as a water-soluble skin cell activating component, whitening agent, moisturizer, thickener, preservative, antioxidant, pH adjuster, film preparation, pigment, colorant, flavor, etc. can be contained as long as the characteristics of the present invention are not impaired.

Other features of the invention will become apparent in the course of the following descriptions of exemplary embodiments which are given for illustration of the invention and are not intended to be limiting thereof.

### EXAMPLES

In each of the following Examples and Comparative Examples, “%” means wt %.

In the Examples and Comparative Examples of the present invention, medium-chain triglyceride (MCT), palm oil, and coconut oil were used as fats and oils. The fatty acid compositions thereof are shown in Table 1.

TABLE 1

| | | fats and oils | | | | | | | |
|---|---|---|---|---|---|---|---|---|---|
| | | medium-chain triglyceride (MCT) | | | | | palm oil | coconut oil | |
| | | product name | | | | | | | |
| | | COCONARD | COCONARD | COCONARD | COCONARD | Liponate | | WHITE | Coconut |
| | | RK | MT | MT-N | ML | GC | RPO | SNOW | Oil #76 |
| | | manufacturer | | | | | | | |
| fatty acid | | | | | | | UEDA- | UEDA- | Welch |
| composition | | Kao | Kao | Kao | Kao | Lipo | SEIYU | SEIYU | Holme & |
| (%) | | Corporation | Corporation | Corporation | Corporation | Chemicals | CO., LTD. | CO., LTD. | Clark |
| caproic acid | 6-0 | 0.5 | 0.3 | 0.1 | 1.9 | | | 0.5 | |
| caprylic acid | 8-0 | 97.3 | 81.6 | 74.7 | 37.6 | 75 | | 0.8 | 7.0 |
| capric acid | 10-0 | 2.2 | 17.4 | 24.7 | 33.9 | 25 | | 6.4 | 7.0 |
| lauric acid | 12-0 | | 0.4 | 0.2 | 20.2 | | 0.3 | 48.5 | 48.0 |
| myristic acid | 14-0 | | | 0.3 | 3.9 | | 1.1 | 17.6 | 17.0 |
| palmitic acid | 16-0 | | | | 1.5 | | 45.1 | 8.4 | 9.5 |
| stearic acid | 18-0 | | | | 0.4 | | 4.7 | 2.5 | 2.5 |
| oleic acid | 18-1 | | | | 0.6 | | 38.8 | 6.5 | 6.5 |
| linoleic acid | 18-2 | | | | | | 9.4 | 1.5 | 1.3 |

### Examples 1-5

### Emulsion Composition

An emulsifier containing decaglycerol monomyristate as a main component (“Sun Soft AB”, manufactured by Taiyo Kagaku Co., Ltd., 20 parts by weight) was mixed with glycerol (“food additive glycerol-S”, manufactured by NOF CORPORATION, 45 parts by weight) at 70° C. to 80° C. and uniformly dissolved. Thereto was added, by small portions, an oil phase prepared in advance by mixing dihydrocapsiate (“DCT”, manufactured by Ajinomoto Co., Inc., 0.075 part by weight) with each of the fats and oils shown in Table 2 (24.93 parts by weight) at 50° C. to 60° C. and dissolving therein, and the mixture was pre-emulsified by TK ROBOMIX (manufactured by PRIMIX Corporation) at 8,000 rpm, 60° C. to 65° C. for 3 minutes. The fatty acid compositions of the fats and oils used are also shown in Table 2. Thereto was added an aqueous phase component obtained by dissolving citric acid (“citric acid (crystal))”, manufactured by Mitsubishi Tanabe Pharma Corporation, 0.05 part by weight) in water (9.95 parts by weight), and the mixture was homogenized by a super high-pressure homogenizer (“Micro Fluidizer”, manufactured by MIZUHO Industrial CO., LTD.) by one treatment (1 pass) at a pressure of 100 MPa to give emulsion compositions of Examples 1 to 5.

### Examples 6-10

### Aqueous Drinks

To aqueous solutions with the compositions shown in Table 3 were added the emulsion compositions of Examples 1 to 5 to 1% content, and 40 mL each of the mixtures was filled in a 100 mL glass bottle and the bottle was tightly sealed. The bottles were sterilized by heating at 80° C. for 20 minutes and cooled with running water to around room temperature to give aqueous drinks of Examples 6 to 10.

TABLE 2

| | | carbon | Ex. 1 | Ex. 2 | Ex. 3 | Ex. 4 | Ex. 5 |
|---|---|---|---|---|---|---|---|
| | | number- | Coconut Oil | Coconut Oil | Coconut Oil | Coconut Oil | Coconut Oil |
| | | double | #76 67%, | #76 33%, | #76 33%, | #76 67%, | #76 80%, |
| | fatty acid | bond | COCONARD | COCONARD | Liponate | Liponate | Liponate |
| | (common name) | number | ML 33% | ML 67% | GC 67% | GC 33% | GC 20% |
| fatty acid | caproic acid | 6-0 | 0.6 | 1.3 | 0.0 | 0.0 | 0.0 |
| composition | caprylic acid | 8-0 | 17.2 | 27.4 | 52.3 | 29.7 | 20.6 |
| (%) | capric acid | 10-0 | 16.0 | 24.9 | 19.0 | 13.0 | 10.6 |
| | lauric acid | 12-0 | 38.7 | 29.5 | 16.0 | 32.0 | 38.4 |
| | myristic acid | 14-0 | 12.6 | 8.3 | 5.7 | 11.3 | 13.6 |
| | palmitic acid | 16-0 | 6.8 | 4.2 | 3.2 | 6.3 | 7.6 |
| | stearic acid | 18-0 | 1.8 | 1.1 | 0.8 | 1.7 | 2.0 |
| | oleic acid | 18-1 | 4.5 | 2.6 | 2.2 | 4.3 | 5.2 |
| | linoleic acid | 18-2 | 0.8 | 0.4 | 0.4 | 0.8 | 1.0 |
| ratio | caprylic acid | 8-0 | 100 | 100 | 100 | 100 | 100 |
| relative to | capric acid | 10-0 | 93 | 91 | 36 | 44 | 51 |
| caprylic | lauric acid | 12-0 | 230 | 110 | 31 | 110 | 190 |
| acid | myristic acid | 14-0 | 73 | 30 | 11 | 38 | 66 |

TABLE 3

| | starting | | addition |
|---|---|---|---|
| component | material name | manufacturer | amount (%) |
| emulsion | — | — | appropriate |
| composition | | | |
| citric acid | food additive | Wako Pure | 0.28 |
| | citric acid | Chemical | |
| | | Industries, Ltd. | |
| sodium citrate | food additive | Wako Pure | 0.090 |
| | sodium citrate | Chemical | |
| | | Industries, Ltd. | |
| high fructose | New Fructo 55 | Showa Sangyo | 1.3 |
| corn syrup | | Co., Ltd. | |
| N-(L-α- | Palsweet Diet | Ajinomoto Co., | 0.011 |
| aspartyl)-L- | | Inc. | |
| phenylalanine 1- | | | |
| methyl ester | | | |
| acesulfame | Sunett D | kirin-food-tech. | 0.012 |
| potassium | | | |
| ion exchange | — | — | amount to |
| water | | | make total |
| | | | 100 |

The distribution of polymerization degree of the constituent polyglycerol in an emulsifier containing decaglycerol monomyristate, which was used for preparation in the above-mentioned Examples, as the main component (“Sun Soft AB”, manufactured by Taiyo Kagaku Co., Ltd.) is shown in Table 4. In the afore-mentioned emulsifier, the ratio of polyglycerol having a polymerization degree of not less than 10 in the constituent polyglycerol was 36.1%. The polymerization degree distribution of the constituent polyglycerol was determined by high-performance liquid chromatography mass spectrometry (LC/MS) under the following analysis conditions.

LC/MS analysis conditions.
ionization mode: APCI, negative
measurement range: 90-2000
column: TSKgel α-2500 (7.8×300 mm)
temperature: 40° C.
eluent: water/acetonitrile 7/3
flow: 0.8 mL, 100 ppm
analysis time: 20 min

TABLE 4

| | constituent polyglycerol/content (%) of Sun Soft AB | | | | | | | | | | | | | | | | |
|---|---|---|---|---|---|---|---|---|---|---|---|---|---|---|---|---|---|
| | degree of polymerization | | | | | | | | | | | | | | | | |
| structure | 1 | 2 | 3 | 4 | 5 | 6 | 7 | 8 | 9 | 10 | 11 | 12 | 13 | 14 | 15 | 16 | total |
| chain | 0.8 | 1.8 | 2.4 | 3.7 | 5.7 | 7.3 | 8.8 | 10.8 | 12 | 11.3 | 8.4 | 5.6 | 3.7 | 2 | 0.8 | 0.2 | 85.3 |
| cyclic | 0 | 0 | 0.2 | 0.3 | 0.4 | 0.6 | 0.8 | 1.1 | 1.1 | 1.1 | 1.1 | 0.8 | 0.7 | 0.3 | 0.1 | 0 | 8.6 |
| unknown | | | | | | | | | | | | | | | | | 6.1 |

*In Table, the chain structure shows linear or branched chain structure.

### Comparative Examples 1 to 9

### Emulsion Compositions

The above-mentioned emulsifier containing decaglycerol monomyristate as the main component (“Sun Soft AB”, manufactured by Taiyo Kagaku Co., Ltd., 20 parts by weight) was mixed with glycerol (“food additive glycerol-S”, manufactured by NOF CORPORATION, 55 parts by weight) at 70° C. to 80° C. and uniformly dissolved. Thereto was added, by small portions, an oil phase prepared in advance by mixing dihydrocapsiate (“DCT”, manufactured by Ajinomoto Co., Inc., 0.045 part by weight) with each of the fats and oils shown in Table 5 (14.96 parts by weight) at 50° C. to 60° C. and dissolved therein, and the mixture was pre-emulsified by TK ROBOMIX (manufactured by PRIMIX Corporation) at 8,000 rpm, 60° C. to 65° C. for 3 minutes. The fatty acid compositions of the fats and oils used are also shown in Table 5. Thereto was added an aqueous phase component obtained by dissolving citric acid (“citric acid (crystal))”, manufactured by Mitsubishi Tanabe Pharma Corporation, 0.05 part by weight) in water (9.95 parts by weight), and the mixture was homogenized by a super high-pressure homogenizer (“Micro Fluidizer”, manufactured by MIZUHO Industrial CO., LTD.) by one treatment (1 pass) at a pressure of 100 MPa to give emulsion compositions of Comparative Examples 1-9.

### Comparative Examples 10-18

### Aqueous Drinks

To aqueous solutions with the compositions shown in Table 3 were added the emulsion compositions of Comparative Examples 1-9 to 1% content, and 40 mL each of the mixtures was filled in a 100 mL glass bottle and the bottle was tightly sealed. The bottles were sterilized by heating at 80° C. for 20 minutes and cooled with running water to around room temperature to give aqueous drinks of Comparative Examples 10-18.

TABLE 5

| | | carbon | | | | |
|---|---|---|---|---|---|---|
| | | number- | Comp. | Comp. | Comp. | Comp. |
| | | double | Ex. 1 | Ex. 2 | Ex. 3 | Ex. 4 |
| | common | bond | COCONARD | COCONARD | COCONARD | COCONARD |
| | name | number | RK 100% | MT 100% | MT-N 100% | ML 100% |
| fatty acid | caproic acid | 6-0 | 0.5 | 0.3 | 0.1 | 1.9 |
| composition | caprylic acid | 8-0 | 97.3 | 81.6 | 74.7 | 37.6 |
| (%) | capric acid | 10-0 | 2.2 | 17.4 | 24.7 | 33.9 |
| | lauric acid | 12-0 | | 0.4 | 0.2 | 20.2 |
| | myristic acid | 14-0 | | | 0.3 | 3.9 |
| | palmitic acid | 16-0 | | | | 1.5 |
| | stearic acid | 18-0 | | | | 0.4 |
| | oleic acid | 18-1 | | | | 0.6 |
| | linoleic acid | 18-2 | | | | |
| ratio | caprylic acid | 8-0 | 100 | 100 | 100 | 100 |
| relative to | capric acid | 10-0 | 2 | 21 | 33 | 90 |
| caprylic | lauric acid | 12-0 | 0 | 0.49 | 0.27 | 54 |
| acid | myristic acid | 14-0 | 0 | 0 | 0.4 | 10 |

| | | | | | Comp. | Comp. |
|---|---|---|---|---|---|---|
| | | Comp. | Comp. | Comp. | Ex. 8 | Ex. 9 |
| | | Ex. 5 | Ex. 6 | Ex. 7 | WHITE | WHITE |
| | | RPO 67%, | RPO 33%, | WHITE | SNOW 67%, | SNOW 33%, |
| | common | COCONARD | COCONARD | SNOW | COCONARD | COCONARD |
| | name | RK 33% | RK 67% | 100% | RK 33% | RK 67% |
| fatty acid | caproic acid | 0.2 | 0.3 | 0.5 | 0.5 | 0.5 |
| composition | caprylic acid | 32.4 | 64.9 | 0.8 | 33.0 | 65.1 |
| (%) | capric acid | 0.7 | 1.5 | 6.4 | 5.0 | 3.6 |
| | lauric acid | 0.2 | 0.1 | 48.5 | 32.3 | 16.2 |
| | myristic acid | 0.7 | 0.4 | 17.6 | 11.7 | 5.9 |
| | palmitic acid | 30.1 | 15.0 | 8.4 | 5.6 | 2.8 |
| | stearic acid | 3.1 | 1.6 | 2.5 | 1.7 | 0.8 |
| | oleic acid | 25.9 | 12.9 | 6.5 | 4.3 | 2.2 |
| | linoleic acid | 6.3 | 3.1 | 1.5 | 1.0 | 0.5 |
| ratio | caprylic acid | 100 | 100 | 100 | 100 | 100 |
| relative to | capric acid | 2.3 | 2.3 | 800 | 15 | 5.5 |
| caprylic | lauric acid | 0.62 | 0.15 | 6063 | 98 | 25 |
| acid | myristic acid | 2.3 | 0.57 | 2200 | 36 | 9 |

### Comparative Example 19

### Emulsion Composition

An emulsifier containing decaglycerol monomyristate as the main component (“Sun Soft AB”, manufactured by Taiyo Kagaku Co., Ltd., 20 parts by weight) was mixed with glycerol (“food additive glycerol-S”, manufactured by NOF CORPORATION, 45 parts by weight) at 70° C. to 80° C. and uniformly dissolved. Thereto was added, by small portions, an oil phase prepared in advance by mixing dihydrocapsiate (“DCT”, manufactured by Ajinomoto Co., Inc., 0.075 part by weight) with each of the fats and oils shown in Table 6 (24.93 parts by weight) at 50° C. to 60° C. and dissolved therein, and the mixture was pre-emulsified by TK ROBOMIX (manufactured by PRIMIX Corporation) at 8,000 rpm, 60° C. to 65° C. for 3 minutes. The fatty acid compositions of the fats and oils used are also shown in Table 6. Thereto was added an aqueous phase component obtained by dissolving citric acid (“citric acid (crystal))”, manufactured by Mitsubishi Tanabe Pharma Corporation, 0.05 part by weight) in water (9.95 parts by weight), and the mixture was homogenized by a super high-pressure homogenizer (“Micro Fluidizer”, manufactured by MIZUHO Industrial CO., LTD.) by one treatment (1 pass) at a pressure of 100 MPa to give the emulsion composition of Comparative Example 19.

### Comparative Example 20

### Aqueous Drink

To an aqueous solution with the composition shown in the above-mentioned Table 3 was added the emulsion composition of Comparative Example 19 to 1% content, and 40 mL of the mixture was filled in a 100 mL glass bottle and the bottle was tightly sealed. The bottle was sterilized by heating at 80° C. for 20 minutes and cooled with running water to around room temperature to give the aqueous drink of Comparative Example 20.

TABLE 6

| | | | Comparative |
|---|---|---|---|
| | | carbon number - | Example 19 |
| | fatty acid | double bond | Coconut Oil #76 |
| | (common name) | number | 100% |
| fatty acid | caproic acid | 6-0 | |
| composition | caprylic acid | 8-0 | 7.0 |
| (%) | capric acid | 10-0 | 7.0 |
| | lauric acid | 12-0 | 48.0 |
| | myristic acid | 14-0 | 17.0 |
| | palmitic acid | 16-0 | 9.5 |
| | stearic acid | 18-0 | 2.5 |
| | oleic acid | 18-1 | 6.5 |
| | linoleic acid | 18-2 | 1.3 |
| ratio | caprylic acid | 8-0 | 100 |
| relative to | capric acid | 10-0 | 100 |
| caprylic | lauric acid | 12-0 | 690 |
| acid | myristic acid | 14-0 | 240 |

The light transmission rate at a wavelength of 600 nm of the emulsion compositions of the above-mentioned Examples and Comparative Examples was measured. The measurement method of the transmission rate is as follows. Each emulsion composition of Examples 1-5 and Comparative Examples 1-9 and 19 was diluted with ion exchange water to 1%, and the light transmission rate at a wavelength of 600 nm was measured by a spectrophotometer (self-recording spectrophotometer; type U-3210, manufactured by Hitachi, Ltd.), using ion exchange water as a control.

The relationship between the above-mentioned transmission rate and the content of the oil phase component in aqueous drinks or ion exchange water follows the Lambert-Beer law.

y₂=(y₁/100)^{(x2/x1)}×100

wherein x₁ and x₂ represent the contents of the oil phase component in aqueous drinks or ion exchange water, and y₁ and y₂ represent the transmission rates. The content of the oil phase component in aqueous drinks or ion exchange water is a value obtained by multiplying the content of the oil phase component in the emulsion composition by the concentration of the emulsion composition added to an aqueous drink or ion exchange water. Therefore, once the transmission rate of an aqueous drink or ion exchange water added with a certain emulsion composition at a certain ratio is known, the transmission rate by the addition at a different ratio can also be calculated. As for the emulsion compositions of Comparative Examples 1-9, the transmission rate of a diluted solution having a content of the oil phase component of 0.25% was calculated from the transmission rate of a solution diluted with 1% ion exchange water (oil phase component content 0.15%). The results are shown in Table 7. When either the measured value of the transmission rate when diluted to contain 0.25% of an emulsion composition as an oil phase component in an aqueous drink or ion exchange water, or the calculated value by the above-mentioned formula was 90% or above, the transparency by addition to an aqueous phase system was judged to be suitable for the object of the present invention.

TABLE 7

| sample | | transmission rate (%) |
|---|---|---|
| Example | 1 | 92 |
| | 2 | 92 |
| | 3 | 93 |
| | 4 | 93 |
| | 5 | 93 |
| Comparative | 1 | 75 |
| Example | 2 | 79 |
| | 3 | 79 |
| | 4 | 95 |
| | 5 | 95 |
| | 6 | 95 |
| | 7 | 95 |
| | 8 | 97 |
| | 9 | 95 |
| | 19 | 92 |

From Table 7, it is seen that all the emulsion compositions of Examples 1-5 of the present invention showed a transmission rate of 90% or above when diluted with ion exchange water such that the oil phase component content was 0.25%, and were suitable for the object of the present invention in terms of the transparency when added to an aqueous phase system. In contrast, of the emulsion compositions of Comparative Examples, wherein the fatty acid composition of the fats and oils constituting the oil phase is not within the composition ratio range of the present invention, the emulsion compositions of Comparative Examples 1-3 were calculated to show a transmission rate of an aqueous drink (oil phase component content 0.25%) of less than 90%, and judged to be unsuitable for the object of the present invention in terms of the aforementioned transparency.

The emulsion stability of each emulsion composition prepared in the Examples and Comparative Examples was evaluated by the following method. The emulsion compositions were mixed well and separately dispensed by 2 g to a glass bottle. To avoid evaporation of water during preservation, the bottle was tightly sealed by tightly wrapping the lid part with a para film. The glass bottles were placed in the shade and preserved at 37° C. (acceleration condition), 5° C. (chilled condition), and 24° C. (distribution condition) for 1 week, and the light transmission rate (%) at a wavelength of 600 nm and average particle size (nm) were measured before and after the preservation. The aforementioned transmission rate was measured or calculated in the same manner as above, and the average particle size was measured by the method shown below. That is, each emulsion composition was diluted with ion exchange water to 1%, and the average particle size was measured by a particle size analyzer (dynamic light scattering type particle size distribution measuring instrument “NICOMP380”; manufactured by Particle Sizing Systems). The emulsion compositions were evaluated and graded in 10 levels by giving 1 to 10 points based on the state where changes in the transmission rate and average particle size are small during the preservation and a stable emulsion was maintained. The criteria of 1, 2, 7, and 10 points are shown below. To achieve the object of the present invention in terms of the emulsion stability, the passing point was set to 1. The results are shown in Table 8.

### Evaluation Criteria:

1 point; almost no changes in average particle size and transmission rate, and stable emulsion is maintained
2 points; slight change in average particle size or transmission rate
7 points; turbidity is visually observed after preservation at 24° C. for 1 week
10 points; separation, oil delamination, and high turbidity are confirmed from immediately after preparation of emulsion composition

TABLE 8

| | | | Example | | | | | Comparative Example | | | | | | | | | |
|---|---|---|---|---|---|---|---|---|---|---|---|---|---|---|---|---|---|
| evaluation item | | | 1 | 2 | 3 | 4 | 5 | 1 | 2 | 3 | 4 | 5 | 6 | 7 | 8 | 9 | 19 |
| transmission | before | — | 95 | 95 | 96 | 96 | 96 | 84 | 87 | 87 | 97 | 97 | 97 | 97 | 98 | 97 | 95 |
| rate (%) | preservation | | | | | | | | | | | | | | | | |
| | after | 5° C. | 95 | 95 | 95 | 95 | 95 | 74 | 78 | 80 | 96 | 95 | 97 | 97 | 96 | 96 | 95 |
| | preservation | 24° C. | 95 | 95 | 95 | 96 | 95 | 44 | 62 | 68 | 95 | 98 | 99 | 97 | 97 | 96 | 95 |
| | | 37° C. | 95 | 95 | 93 | 94 | 94 | 6 | 14 | 21 | 80 | 99 | 99 | 97 | 96 | 91 | 95 |
| particle | before | — | 54 | 52 | 52 | 52 | 52 | 81 | 78 | 74 | 44 | 50 | 47 | 47 | 43 | 45 | 55 |
| size (nm) | preservation | | | | | | | | | | | | | | | | |
| | after | 5° C. | 52 | 51 | 55 | 52 | 51 | 104 | 95 | 88 | 46 | 54 | 54 | 47 | 44 | 52 | 54 |
| | preservation | 24° C. | 53 | 55 | 55 | 51 | 53 | 153 | 127 | 120 | 50 | 48 | 51 | 47 | 44 | 48 | 53 |
| | | 37° C. | 56 | 58 | 57 | 54 | 53 | 260 | 218 | 199 | 91 | 48 | 50 | 49 | 49 | 66 | 56 |

| | | | | | | | | | | | | | | | |
|---|---|---|---|---|---|---|---|---|---|---|---|---|---|---|---|
| emulsion stability | 1 | 1 | 1 | 1 | 1 | 8 | 7 | 7 | 4 | 2 | 2 | 1 | 2 | 3 | 1 |
| (evaluation point) | | | | | | | | | | | | | | | |

From Table 8, it is seen that the emulsion compositions of Examples 1-5 of the present invention showed almost no changes in the transmission rate and particle size, and showed good emulsion stability, even after preservation at any temperature. In contrast, the emulsion compositions other than Comparative Examples 7 and 19 showed decreased transmission rate or increased particle size over time, and did not show sufficient emulsion stability.

Subsequently, the stability of dihydrocapsiate (DCT) in respective aqueous drinks of Examples 6-10 and Comparative Examples 10-18 and 20 was evaluated by the following method. First, each aqueous drink was placed in the shade and preserved under the conditions of 44° C., relative humidity 78%, for 2 weeks. The DCT contents before and after preservation were calculated from the following quantitative analysis, and the residual rate was calculated from the analysis value. The residual rate was calculated by the following formula.

Residual rate (%)=DCT content after preservation/DCT content before preservation×100

The quantitative analysis method of the dihydrocapsiate content was as described below. Each aqueous drink (4 g) was poured into a 20 mL measuring flask, and measured up by adding a mixed solvent of ethyl acetate:methanol=6:4. The measured-up solution was filtered with a filter (0.45 μm mesh) and analyzed by high performance liquid chromatography (HPLC).

To achieve the object of the present invention, an emulsion composition that satisfied the following criteria passed. When an emulsion composition did not show a DCT residual rate satisfying the following criteria, the emulsion composition was rejected due to the lack of DCT stability.

### Stability Criteria:

when 0.15% of emulsion composition-derived oil phase component is contained in aqueous drink:DCT residual ratio not less than 82.0%
when 0.25% of emulsion composition-derived oil phase component is contained in aqueous drink:DCT residual ratio not less than 85.0%

The results are shown in Table 9.

TABLE 9

| | Example | | | | | Comparative Example | | | | | | | | | |
|---|---|---|---|---|---|---|---|---|---|---|---|---|---|---|---|
| | 6 | 7 | 8 | 9 | 10 | 10 | 11 | 12 | 13 | 14 | 15 | 16 | 17 | 18 | 20 |
| oil phase | 0.25 | | | | | 0.15 | | | | | | | | | 0.25 |
| component content | | | | | | | | | | | | | | | |
| (%) in aqueous | | | | | | | | | | | | | | | |
| drinks | | | | | | | | | | | | | | | |
| DCT residual | 88.6 | 89.1 | 87.5 | 87.2 | 88.9 | 85.0 | 84.5 | 85.6 | 85.2 | 83.8 | 84.2 | 78.3 | 81.7 | 84.1 | 84.9 |
| ratio (%) in | | | | | | | | | | | | | | | |
| aqueous drinks | | | | | | | | | | | | | | | |

As shown by the results presented in Table 9, the aqueous drinks of Examples 6-10 which were preserved at 44° C., relative humidity 78%, for 2 weeks satisfied the above-mentioned criteria of the dihydrocapsiate residual ratio. In contrast, the aqueous drinks of Comparative Examples 16 and 20 containing the emulsion compositions of Comparative Examples 7 and 19, which showed emulsion stability in the above-mentioned Table 8, did not satisfy the above-mentioned criteria of the stability of dihydrocapsiate contained in aqueous drinks.

### INDUSTRIAL APPLICABILITY

As described above, the present invention provides an emulsion composition which is superior in emulsion stability after long-term preservation, is capable of stably containing an oil-soluble component having various physiological functions, affords good transparency when added to an aqueous phase system, and is suitable for application to foods and drinks such as aqueous drinks and the like, pharmaceutical products such as internal liquid, liquid and the like, and cosmetic agents such as skin lotion, beauty essence and the like.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An emulsion composition, comprising:
(A) an oil phase component, comprising at least one oil-soluble component and at least one fat or oil;
(B) at least one polyglycerol fatty acid ester; and
(C) an aqueous phase component,
wherein
said (A) at least one fat and oil comprises caprylic acid, capric acid, lauric acid, and myristic acid in weight ratios of:
capric acid:caprylic acid of 20 to 97:100,
lauric acid:caprylic acid of 28 to 6000:100, and
myristic acid:caprylic acid of 11 to 2100:100.

2. An emulsion composition according to claim 1, which comprises 1 to 2000 parts by weight of said (A) oil phase component relative to 100 parts by weight of said (B) polyglycerol fatty acid ester.

3. An emulsion composition according to claim 1, wherein a water dispersion obtained by dispersing said emulsion composition in water such that said (A) oil phase component is contained in an amount of 0.25 wt %, based on the total weight of said dispersion, exhibits a light transmission rate of not less than 90% at a wavelength of 600 nm.

4. An emulsion composition according to claim 1, wherein said (B) polyglycerol fatty acid ester comprises, as a main component, a polyglycerol monomyristate which is an ester of a polyglycerol containing not less than 30 wt % of polyglycerol having a degree of polymerization of not less than 10 and a fatty acid containing not less than 90 wt % of myristic acid.

5. An emulsion composition according to claim 1, wherein said (A) oil-soluble component comprises one or more capsinoid compound.

6. An emulsion composition according to claim 5, wherein said one or more capsinoid compound is selected from the group consisting of capsiate, dihydrocapsiate, and nordihydrocapsiate.

7. An emulsion composition according to claim 2, wherein said (A) oil-soluble component comprises one or more capsinoid compound.

8. An emulsion composition according to claim 7, wherein said one or more capsinoid compound is selected from the group consisting of capsiate, dihydrocapsiate, and nordihydrocapsiate.

9. An emulsion composition according to claim 3, wherein said (A) oil-soluble component comprises one or more capsinoid compound.

10. An emulsion composition according to claim 9, wherein said one or more capsinoid compound is selected from the group consisting of capsiate, dihydrocapsiate, and nordihydrocapsiate.

11. An emulsion composition according to claim 4, wherein said (A) oil-soluble component comprises one or more capsinoid compound.

12. An emulsion composition according to claim 11, wherein said one or more capsinoid compound is selected from the group consisting of capsiate, dihydrocapsiate, and nordihydrocapsiate.

13. A food or drink comprising 0.001 wt % to 10 wt %, based on the total weight of said food or drink, of an emulsion composition according to claim 1.

14. A food or drink according to claim 13, wherein said (A) oil-soluble component is one or more capsinoid compound.

15. A food and drink of claim 14, wherein said one or more capsinoid compound is selected from the group consisting of capsiate, dihydrocapsiate, and nordihydrocapsiate.

16. A food or drink comprising 0.001 wt % to 10 wt %, based on the total weight of said food or drink, of an emulsion composition according to claim 2.

17. A food or drink according to claim 16, wherein said (A) oil-soluble component is one or more capsinoid compound.

18. A food and drink of claim 17, wherein said one or more capsinoid compound is selected from the group consisting of capsiate, dihydrocapsiate, and nordihydrocapsiate.
